(19) United States
(12) Patent Application Publication
(10) Pub. No.: US 2013/0066370 A1
(43) Pub. Date: Mar. 14, 2013

(54) SURGICAL SUTURES AND METHODS OF MAKING AND USING SAME

(71) Applicants: Richard H. Spedden, Clarksville, MD (US); Lew C. Schon, Baltimore, MD (US); Margaret E. Thorpe, Baltimore, MD (US)

(72) Inventors: Richard H. Spedden, Clarksville, MD (US); Lew C. Schon, Baltimore, MD (US); Margaret E. Thorpe, Baltimore, MD (US)

(73) Assignee: The Stem Cell Suture Company, LLC

(21) Appl. No.: 13/604,450

(22) Filed: Sep. 5, 2012

Related U.S. Application Data
(60) Provisional application No. 61/531,424, filed on Sep. 6, 2011.

(51) Int. Cl. A61L 17/08

(52) U.S. Cl. 606/231

(57) ABSTRACT

Provided in one embodiment is a suture, comprising: a porous interior component comprising a volume of pores or interstices, the volume comprising a binding surface; and a first number of biological cells dispersed in the volume; wherein the first number of biological cells in the volume is greater than a threshold number representing a second number of the biological cells at 100% confluence on the binding surface.

### RELATED APPLICATION

This application claims priority from U.S. Provisional Application Ser. No. 61/531,424, filed Sep. 6, 2011, which is hereby incorporated by reference in its entirety.

### BACKGROUND

Surgical sutures find common use in a broad range of medical procedures to hold tissues of the human body together after they have been severed by injury or surgery. In addition to serving as tissue fasteners, sutures and other elongate, thread-like medical devices can serve as a tissue scaffold or structural support for or during the growth of new tissue at a target tissue site in a patient, such as in tendon repair.

Rarely is a sufficient concentration and density of bioactive material maintained at the target site over the requisite period of time needed for the suture (and the bioactive material associated therewith) to exert its beneficial effect. Though this effect may be countered by providing the suture surface with an overabundance of bioactive material, if care is not taken, high local concentrations of bioactive material can result in deleterious, even toxic effects. Moreover, given the high cost of manufacture for certain bioactive materials, particularly natural and synthetic growth factors, this is not a cost effective solution.

### SUMMARY

In view of the foregoing, the present inventors have recognized and appreciated the advantages of a surgical suture and/or porous medical implant loaded with a controlled high density of biological cells and/or having varying hydrophobicity in different portions thereof.

Accordingly, provided in one embodiment is a suture, comprising: a porous interior component comprising a volume of pores or interstices, the volume comprising a binding surface; and a first number of biological cells dispersed in the volume; wherein the first number of biological cells in the volume is greater than a threshold number representing a second number of the biological cells at 100% confluence on the binding surface.

Provided in another embodiment is a method of implanting a suture in a subject in need thereof, comprising: exposing a suture, which comprises a porous interior component comprising a volume of pores or interstices, the volume comprising a binding surface, to a medium comprising a concentration of biological cells, such that a first number of the biological cells are dispersed in the volume of the pores or interstices; and implanting the suture into the subject; wherein the concentration of the biological cells is sufficiently high such that the first number of the biological cells is greater than a threshold number representing a second number of the biological cells at 100% confluence on the binding surface.

Provided in another embodiment is a suture, comprising: a porous interior component comprising pores or interstices; and a first number of biological cells dispersed in a plurality of the pores or interstices; wherein the suture has a plurality of portions having multiple levels of hydrophobicity.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 provides a schematic showing, as illustration, that due to their larger surface areas in one embodiment, fewer adherent mesenchymal stem cells (“MSCs”) can inhibit the suture than unattached (spherical) MSCs because there is physically not enough room for adherent MSCs to fit on the suture.

FIG. 2 provides a schematic in one embodiment illustrating that once placed in tissue, unbound MSC may migrate out of the pores or interstices of the suture and into the surrounding tissues; once released at the site of repair the MSCs could undergo differentiation and secrete cytokines to aid in tissue healing and recruitment of host cells.

FIG. 3 provides photomicrographs (10×) of tendon repair sites comparing at different time points the effects of a pre-existing, conventional suture repair, a suture repair with stem cells injected into the tissue in the vicinity of the repair and a suture with a high loading level of stem cells in one embodiment described herein.

FIG. 4 shows comparison data for the number of cells delivered to the site of repair for a suture with cells directly cultured on a suture for 3 and 5 days and for a suture overloaded with mesenchymal stem cells (“MSCs”) in one embodiment.

### DETAILED DESCRIPTION

Following below are more detailed descriptions of various concepts related to, and embodiments of, inventive surgical sutures and porous medical implants loaded with a high density of biological cells. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

One embodiment provides methods and devices for an improved stem cell bearing surgical suture, particularly the methods and devices, wherein a medical implant device, such as a suture (e.g., a surgical suture), is loaded with biological cells (e.g., stem cells), at a concentration above that achievable in a sustainable culture or in vivo. In one embodiment, a surgical suture is loaded with a quantity of cells in excess of the number of cells that may be supported by the available binding surfaces of the implant at cellular confluence.

The term “medical device” herein may encompass both devices intended for temporary introduction (for example, bioerodible sutures or tissue scaffolds), as well as devices intended for long term or permanent insertion (for example, artificial ligaments or tendons). In one embodiment, the term “medical device” may refer to any apparatus, appliance, instrument, implement, material, machine, contrivance, implant, in vitro reagent, or other similar or related article including a component party or accessory, which is intended for the diagnosis, prevention, monitoring, treatment or alleviation of disease, injury or handicap. The term may also encompass any article intended to affect the structure or function of the body of humans or other animals, and which does not achieve its principal intended action in or on the body exclusively by pharmacological, immunological or metabolic means, but which may be assisted in its function by such means. Illustrative examples of medical devices include, but are not limited to, needles, catheters (e.g., intravenous, urinary, and vascular catheters), stents, shunts (e.g., hydrocephalus shunts, dialysis grafts), tubes (e.g., myringotomy tubes, tympanostomy tubes), implants (e.g., breast implants, intraocular lens), prosthetics, and artificial organs, as well as cables, leads, wires, and electrodes associated therewith (e.g., leads for pace makers and implantable defibrillators, bipolar and monopolar RF electrodes, vascular guidewires). Also contemplated are devices such as wound dressings, sutures, staples, anastomosis devices, vertebral disks, bone pins, suture anchors, hemostatic barriers, clamps, screws, plates, clips, vascular implants, tissue adhesives and sealants, tissue scaffolds, various types of dressings, bone substitutes, ligament or tendon implant devices, intraluminal devices, vascular supports, and other body contacting devices that may benefit from incorporation with therapeutic materials such as therapeutic agents, bioactive molecules, and biological cells or tissues.

The term “suture” herein may refer to an elongate, generally tubular and thread-like medical device provided with a proximal end, a distal end and a longitudinal axis, where the length along the longitudinal axis is equal to or greater than twice the length on any other axis, and where the device is flexible along the longitudinal axis. In some embodiments, sutures are used to join tissue or medical prosthesis. In some embodiments, sutures may also be employed for the immobilization and delivery of therapeutic materials to a tissue site or as a scaffold for tissue growth.

The term “structural properties” in the context of a suture herein may refer to those properties that permit the device to withstand tensile forces in the longitudinal, circumferential, or radial directions without premature failure or a level of yield that would prevent the device from functioning in its intended manner over the anticipated functional life of the device.

The term “porous” herein may refer to voids or openings of functionally relevant size in the matrix of materials, such as those in the sheath and/or core in the interior component of one embodiment. In one embodiment, the voids may refer to pores or interstices. In the context of openings in or passageways through or out of the sheath or the core, the functionally relevant size is the size that permits or inhibits passage of cells, therapeutic materials or other materials of the construct that might otherwise have mobility. In one embodiment wherein biological cells are involved, the functionally relevant size to permit passage of single cells may refer to a size greater than the cell size, which is typically in the 5 to 50 μm range. In another embodiment wherein one objective is to inhibit flow (and thus complete prevention of flow is not needed), a much larger diameter opening may be employed to provide the desired result. This may be particularly effective in the case where any interstitial fluid (or cell carrier medium) has a viscosity sufficient to retard flow, the flow path is relatively long or convoluted, or where other materials present in the fluid are of a size or density to impede flow. In some embodiments, particularly with a braided or woven matrix (as is common in sutures or in the sheath described in one embodiment herein), the openings in a porous medium, such as a sheath, need not be round—in many cases, the geometry may vary with the type of stress that the medium is put under; in such cases, the relevant dimension may be the minimum dimension across the opening. The pores or interstices may have a size that permits retention of biological cells within the pores or interstices. Alternatively (or additionally), the size may be suitable for migration or passage of cells from one region or area of the suture to another.

Relevant porosity, in the case of bulk medium, such as in the core of a suture which comprises biological cells, may refer to the voids which are of a size to include therapeutic materials in a total quantity in the medium to have therapeutic effect, or the voids of a size necessary to allow inclusion of biological cells in the medium. In one embodiment, in the case of a suture core, relevant voids may include the space between fibers in a multifilament core, the space between the core and the sheath, the space between particles is a particulate-bearing core, or voids in the actual bulk material in the core (e.g., a sponge-like material). Mygind, T., et al. (“Mesenchymal stem cell ingrowth and differentiation on coralline hydroxyapatite scaffolds,” Biomaterials, 28 (6): 1036-1047 (February 2007)) have indicated that there are benefits to stem cell proliferation and differentiation with pore sizes in the 100 to 500 μm range. Pore sizes outside of this range may also be employed, depending on the application.

Medical devices, particularly surgical sutures, may be fabricated from a wide range of materials. The materials may be biodegradable and/or biocompatible. Biodegradable polymers may include, but are not limited to, the polyester family, such as polyglycolides and polylactides, the polyorthoesters family, the polyanhydrides family, the polyphosphazenes family and polyhydroxyalkanoates. More specific examples of biocompatible polymers include: polyesters of [alpha]-hydroxycarboxylic acids, such as poly(L-lactide) (PLLA) and polyglycolide (PGA); poly-p-dioxanone (PDO); polycaprolactone (PCL); polyvinyl alcohol (PVA); polyethylene oxide (PEO); polymers disclosed in U.S. Pat. Nos. 6,333,029 and 6,355,699; and any other bioresorbable and biocompatible polymer, co-polymer or mixture of polymers or co-polymers that are utilized in the construction of medical implant devices.

The materials may be natural materials—e.g., collagen and silk. The materials may alternatively be synthetic materials. In one embodiment, some of these biocompatible materials may be conjugated with bioactive molecules (e.g., growth factors) using any suitable technologies depending on the application. The biodegradable materials may also include materials which may or may not be suited to conjugation with bioactive molecules. In some embodiments, bioactive molecules may be incorporated into the construct of a medical or surgical device construct without necessitating conjugation or chemical bonding with the device material. In addition, as new biocompatible, bioresorbable materials are developed, at least some of them may be useful materials in the embodiments described herein. It should be understood that the above materials are identified by way of example only and are not limited to any particular material unless expressly called for in the claims.

Bone marrow for clinical use may be obtained as an aspirate extracted from a target patient's bone using a syringe-type device. Often the hip bone is used as a source, in part because of its large size and proximity to the surface of the body. In some applications, the bone marrow is used without modification, but in many cases some form of separation technology, such as centrifugation, may be used to concentrate the desired fraction of the bone marrow. Bioactive molecules, including cytokines such as growth factors, are often the target of this separation process. Stem cells, progenitor cells, or other cells may also be desired targets. Cells and molecules of interest may also be obtained from adipose, also fat, tissue, as well as from various fluids in the body. Other suitable tissues may include muscle and nerve tissue and tissues associated with the reproductive process are also of particular interest. Material extracted from the patient may have several advantages over other sources, such as inherent biocompatibility, potentially lower cost, and providing a broader spectrum of useful compounds that might have synergistic effects. In current surgical practice, bone marrow derivatives are typically reintroduced into the body by injection by syringe into an area of desired activity. Often a porous retention medium, such as a collagen sponge, may be used to retain the material in the area.

The term “cell” or “biological cell” may refer to any cell capable of peforming useful biological functions in a living organism, particularly replication to form a tissue structure. Biological cells may include cells from the intended host organism or those from a donor organism. Biological cells may include cells from recombinant or genetic engineering techniques. The term as used herein may include stem cells (e.g., mesenchymal stem cells), progenitor cells, and fully differentiated cells. Examples include one or more of the following: chondrocytes; fibrochondrocytes; osteocytes; osteoblasts; osteoclasts; synoviocytes; bone marrow cells; mesenchymal cells; stromal cells; stem cells; embryonic stem cells; precursor cells derived from adipose tissue; peripheral blood progenitor cells; stem cells isolated from adult tissue; genetically transformed cells; a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of precursor cells isolated from adult tissue and other cells; a combination of peripheral blood progenitor cells and other cells; a combination of stem cells isolated from adult tissue and other cells; and a combination of genetically transformed cells and other cells. Any other cells having therapeutic value may be employed as the biological cells described herein.

The term “stem cell” may refer to a generic group of undifferentiated cells that possess the capacity for self-renewal while retaining varying potentials to form differentiated cells and tissues. Stem cells may be totipotent, pluripotent or multipotent. Derivative stem cells that have lost the ability to differentiate also occur and may be referred to as “nullipotent” stem cells. A totipotent stem cell is a cell that has the ability to form all the cells and tissues that are found in an intact organism, including the extra-embryonic tissues (i.e. the placenta). Totipotent cells comprise the very early embryo (8 cells) and have the ability to form an intact organism. A pluripotent stem cell is a cell that has the ability to food all tissues found in an intact organism although the pluripotent stem cell cannot form an intact organism. A multipotent cell has a restricted ability to form differentiated cells and tissues. In one embodiment, adult stem cells are multipotent stem cells and are the precursor stem cells or lineage restricted stem cells that have the ability to form some cells or tissues and replenish senescing or damaged cells/tissues. Additional descriptions of stem cells may be found in WO 08/007,082.

The term “progenitor cell” herein may refer to unipotent or multipotent cells, which comprise the stage of cell differentiation between stem cells and fully differentiated cells.

The term “bioactive molecules” herein may refer to any molecule that has the capacity to interact with a living tissue or system in such a way as to exhibit or induce a biological activity in an organism, tissue, organ or cell, either in vivo, in vitro or ex vivo. The term “bioactive molecule” in one embodiment extends to precursor forms thereof. Precursor proteins, for example BMP precursors, may be inactive until they undergo endoproteolytic cleavage. In some embodiments, precursor proteins may also be included as bioactive molecules.

In some embodiments, bioactive molecules may include peptides that trigger or regulate biological functions. Illustrative examples of bioactive molecules suitable for use herein include, but are not limited to, growth factor proteins, such as TGFβ, BMP-2, FGF and PDGF.

The tell “growth factors” herein may refer to a broad class of bioactive polypeptides that control or regulate a variety of endogenous biological and cellular processes, such as cell-cycle progression, cell differentiation, reproductive function, development, motility, adhesion, neuronal growth, bone morphogenesis, wound healing, immune surveillance and cell apoptosis. Growth factors may operate by binding to specific receptor sites on the surface of target cells. Growth factors may include, but are not limited to, cytokines, chemokines, polypeptide hormones and the receptor-binding antagonists thereof Examples of growth factors may include: Bone Morphogenic Protein (BMP); Transforming growth factor beta (TGF-β); Interleukin-17; Transforming growth factor alpha (TGF-α); Cartilage oligomeric matrix protein (COMP); Cell Density Signaling Factor (CDS); Connective tissue growth factor (CTGF); Epidermal growth factor (EGF); Erythropoietin (EPO); Fibroblast growth factor (FGF); Glial Derived Neurotrophic Factors (GDNF); Granulocyte-colony stimulating factor (G-CSF); Granulocyte-macrophage colony stimulating factor (GM-CSF); Growth differentiation factor (GDF); Myostatin (GDF-8); Hepatocyte growth factor (HGF); Insulin-like growth factor (IGF); Macrophage inhibitory cytokine-1 (MIC-1); Placenta growth factor (PIGF); Platelet-derived growth factor (PDGF); Thrombocyte concentrate (PRP); Thrombopoietin (TPO); Vascular endothelial growth factor (VEGF); Activin and Inhibin; Coagulogen; Follitropin; Gonadotropin and Lutropin; Mullerian Inhibiting Substance (MIS) also called: Anti-Müllerian hormone (AMH) Mülllerian inhibiting factor (MIF) and Müllerian inhibiting hormone (MIH); Nodal and Lefty; and Noggin.

Molecules which regulate, induce or participate in useful biological processes in the body, including those listed above, may be categorized or classified according to their particular structure or function. For example, immunoregulatory proteins secreted by cells of the immune system, such as interleukin and interferon, may be referred to as cytokines. Other categories of regulatory molecules include, but are not limited to: morphogens (e.g., molecules that regulate or control the formation and differentiation of tissues and organs); chemokines (e.g., any of a group of cytokines produced by various cells, as at sites of inflammation, that stimulate chemotaxis in white blood cells such as neutrophils and T cells); hormones (e.g., a product of living cells that circulates in body fluids such as blood and produces a specific, often stimulatory effect on the activity of cells, usually remote from its point of origin); receptors (e.g., a molecule present on a cell surface or in the cell interior that has an affinity for a specific chemical entity, including both endogenous substances such as hormones and ligands as well as foreign materials, such as viral particles, that serves as an intermediary between the stimulating agent and the downstream physiological or pharmacological response thereto; receptor-binding agonists (e.g., a chemical substance capable of combining with a specific receptor on a cell and initiating the same reaction or activity typically produced by the endogenous binding substance (such as a hormone); and receptor-binding antagonists (e.g., a chemical substance that reduces the physiological activity of another chemical substance (such as a hormone) by combining with and blocking one or more receptors associated therewith.

The term “growth factors” herein may also refer to precursor forms of growth factors, which may be inactive until they undergo endoproteolytic cleavage, as well as synthesized and recombinant forms which provide part or all of the same or similar functions as the naturally occurring growth factors. Accordingly, in one embodiment, the molecules described herein may encompass precursors, analogues, and functional equivalents of growth factors, provided the resulting molecules retain some or all of the functions of regulating useful biological processes in the body, such as by binding to specific receptor sites on the surface of target cells associated with the wild-type or endogenous moiety.

The term “therapeutic agents” herein as used herein may refer to any molecule, compound, or composition having therapeutic potential, more particularly pharmaceutical activity. Examples of particularly useful therapeutic and/or pharmaceutical activities may include, but are not limited to, anti-coagulation activity, anti-adhesive activity, anti-microbial activity, anti-proliferative activity, and biomimetic activity.

The term “antimicrobial” herein may refer to any molecule that has the capacity to limit or interfere with the biological function of a bacterial, fungal or viral pathogen or a toxin. Antimicrobial may encompass antibacterial, antibiotics, antiseptics, disinfectants and combinations thereof.

The term “biomimetic” herein may refer to a material which exhibits surface properties, including, but not limited to, molecular structures such as amino acid and carbohydrate sequences, which provide the surface with characteristics, and in particular molecular binding or biological recognition features, which are in common with or provide functional analogues with biological features of biological materials, such as tissue, and in particular cells, which the surface is intended to represent. The term biomimetic in one embodiment refers to that the surface duplicates all functions or binding modalities of the biological material being mimicked. Examples of such structures to be mimicked include pathogen binding proteins and immune recognition sequences (e.g., glycan signatures). Whether a particle moiety possesses the desired biomimetic activity may be assayed using any suitable techniques. For example, one may utilize immunoassay techniques, such as ELISA, to assay the binding activity of a proposed biomimic as compared to endogenous host tissue. Alternatively, one may utilize immune response assays, such the multiplexed chemokine and cytokine assays available through Meso Scale Discovery (MSD) (Gaithersburg, Md.), to assess the risk and assay immunogenic potential of a proposed biomimic as compared to native tissue.

The term “therapeutic materials” herein may refer to any composition that comprises any of the following: therapeutic agents, bioactive molecules, stem cells, progenitor cells or biological cells. The term “bioactive solution” may refer to a liquid composition that comprises, in part, bioactive materials.

The term “tissue” herein may refer to biological tissues. In one embodiment, the term refers to a collection of interconnected cells that perform a similar function within an organism. Four types of tissue generally may be found in the bodies of all animals, including human and lower multicellular organisms such as insects, including epithelium, connective tissue, muscle tissue, and nervous tissue. In one embodiment, the term “tissue” may refer to all biological components including, but not limited to, skin, muscle, nerves, blood, bone, cartilage, tendons, ligaments, and organs composed of or containing same.

The term “bone” may refer to the rigid organs that form part of the endoskeleton of vertebrates and function to move, support, and protect the various organs of the body, produce red and white blood cells and store minerals. One of the types of tissues that make up bones is the mineralized osseous tissue, or bone tissue, which provides bones with rigidity and honeycomb-like three-dimensional internal structure. Other types of tissue found in bones include marrow, endosteum, and periosteum, nerves, blood vessels and cartilage. In one embodiment, cartilage is a dense connective tissue containing collagen fibers and/or elastin fibers that may supply smooth surfaces for the movement of articulating bones. Cartilage is found in many places in the body including the joints, the rib cage, the ear, the nose, the bronchial tubes and the intervertebral discs. There are three main types of cartilage: elastic, hyaline, and fibrocartilage.

The term “isolated,” as in, for example “isolated from biological tissues or cells,” may refer to any process which separates the therapeutic material of interest from the tissue or cell membranes in a manner which preserves the structure and function of therapeutic material of interest. The term “isolated” as used herein may be synonymous with the terms “extracted” and “harvested,” for example. In addition to being isolated, harvested or extracted from natural sources, therapeutic materials suitable for use in the embodiments described herein may also be “derived from” biological sources, for example, synthetically produced or produced by genetically engineered plants and animals, including bacteria and other microbes, in accordance with well-known and conventional techniques. In one embodiment, isolation may also include purification of the biological material from the biological source.

A “viscous material” herein may refer to a flowable or pliable material having either a high coefficient of viscosity or a high measured surface tension or both, whereby either or both of viscous forces and surface tension forces serve to retain the material within a pore or interstitial space to thereby obstruct said pore or space and prevent fluid flow therethrough.

The term “immobilization” of bioactive molecules, such as biological cells, may refer to any “capture” and “retention” mechanisms, ranging from ionic or covalent binding to adsorption or absorption to simple physical capture, including entrapment, entanglement or entrainment.

### Suture Configuration

The materials and methods described herein may enable medical personnel, and more particularly surgical teams, to utilize therapeutic materials that may need immediate use or have restrictive storage requirements, particularly harvested stem cells. The materials may be extracted from the patient who is the intended recipient of the medical procedure (referred to herein as autografts or autogenic tissues); examples include stem, progenitor and other biological cells, bioactive molecules, and other therapeutic materials. The stem, progenitor, and other cells, and bioactive molecules may be derived from any tissue of the body in which the material is present, including bone marrow, adipose tissue, muscle tissue and nerve tissue and any fluids associated with those tissues. Alternately or additionally, the cells and molecules may include, in part, materials derived from other sources, including homologous and heterologous transplant material such as allografts, xenografts (also zenografts), synthetic mimics of tissues, or genetically engineered molecules or cells, and the materials may include bioactive molecules and stem, progenitor and other cells. Alternatively, the cells and molecules described herein may be derived from products of the human or mammalian reproductive system, including autografts, allografts and xenografts of the same. In one embodiment wherein the suture is an autograft, the bioactive materials (e.g., biological cells) may be extracted by the surgical team and introduced into the surgical suture or precursor construct described herein, followed by the reintroduction into the patient (or subject). In one embodiment, this may take place with one single surgical procedure.

The sutures described herein may be surgical sutures. The suture described herein may be employed to provide a structural connection between adjoining tissues. One embodiment provides a suture, comprising: a porous interior component comprising a volume of pores or interstices, the volume comprising a binding surface; and a first number of biological cells dispersed in the volume. The term “binding surface” is described further below. In one embodiment, the first number of biological cells in the volume is greater than a threshold number representing a second number of the biological cells otherwise at 100% confluence on the binding surface of the suture.

The suture, particularly the interior component thereof, may contain biological cells. U.S. application Ser. No. 12/489,557 to Spedden et al provides descriptions of surgical sutures containing biological cells. The suture structures described herein may have any type of configuration and components including what is described in the aforementioned described application.

The suture may have any suitable shape and dimension, depending on the application. In one embodiment, the suture may be cylindrical (or tubular) in shape. The suture may take the form of a monofilament or multifilament material, such filaments including those of approximately cylindrical geometries, as well as other cross-sectional geometries, including film or tape-like geometries. The suture may take the form of a surgical suture, a thread-like tissue scaffold, or a precursor construct. The suture may comprise different components. For example, the suture may have an interior component, which may be porous. In one embodiment, the porous interior component may further comprise a porous interior core; and an exterior component exterior relative to the interior porous core. In an alternative component, the interior component may comprise only a porous interior core and no exterior component. The exterior component of the porous interior component may be of any forms, such as a sheath, coat, sleeve, etc. In one embodiment, the suture may comprise additional components exterior to the interior component; such a component may be referred to as an “outer component,” which is exterior to the interior component. In other words, in the case where the interior component has an exterior component (sheath) covering an interior core, the outer component would be exterior relative to the exterior component.

In one embodiment wherein the interior component comprises both a porous interior core and a sheath exterior relative to the core, the sheath and interior core may take a number of alternate forms. For example, the sheath and core may comprise (e.g., be fabricated from) the same or different materials. In one embodiment, these two components may comprise a mixture of materials, such that the elements of the sheath and core are contiguous or connected. The sheath and/or core components may exhibit varying degrees of biodegradability or be relatively inert in tissue. As described further below, they may have different levels of hydrophobicity (or hydrophilicity).

In one embodiment, the exterior sheath may comprise a matrix of a rapidly absorbed material and a structural filament material of reduced absorbability. Such a construction may have several surprising benefits. For example, a less-porous suture sheath which transitions to a more porous state may be desirable if biological cells are present in the core. The more porous state may allow molecules needed for cell growth or survival to permeate into the body of the suture to reach the cells while still retaining the cells in the desired scaffold configuration. On the other hand, the matrix of rapidly absorbed material and structural filament material may present a relatively smooth surface and less-porous surface while the suture is being handled or introduced into the tissue and then the suture surface may transition to a more porous surface once it is implanted into the subject. In one embodiment, the less-porous surface may present less drag as it is introduced into the tissue. The less-porous surface may also represent an exterior surface that has a reduced tendency of picking up unwanted molecules or biologics prior to introduction into the tissue. The more porous surface, which develops after the suture is implanted in the tissue, may facilitate permitting molecules in the core to diffuse out of the suture for additional therapeutic or antimicrobial purpose. The rapidly absorbed material may include, at least in part, molecules of antimicrobial or therapeutic value, while the structural filament material may maintain the structural or scaffold properties of the construct.

Some surprisingly benefits may include that the matrix of rapidly absorbed material and structural filament material may present a relatively smooth surface when the suture passes through the tissue during implantation. After the rapidly absorbed material begins to dissipate, the suture may exhibit other surface properties, such as surface roughness. Increased roughness may be desirable for reducing the movement of suture in the tissue, thus reducing the tendency of the suture to “saw” or cut, and thus further to scar the tissue. Further, the matrix of rapidly absorbed material and structural filament material may present a surface which does not readily bind to the surrounding tissue during the process of passing the suture through the tissue, and then after the rapidly absorbed material begins to dissipate, structural filament or other material with surface properties which bind to surrounding tissues may be exposed, providing the benefit of fixing the suture in place. The surface may contain any molecule that may be immobilized on a suture surface with appropriate tissue binding properties—e.g., lectins and heparin compounds. A wide range of immobilization techniques for such compounds on a surface of, for example, polymers may be employed.

The matrix of materials forming the sheath of a surgical suture (or precursor construct in some instances) herein may include two different synthetic polymers, a synthetic polymer and a natural material, or two natural materials, any or all of which may optionally be biodegradable. Any suitable techniques to combine these materials into a woven, non-woven, or film-like construct may be employed. In one embodiment, the structural fibers and the more biodegradable fibers are interwoven. In another embodiment, the structural fibers and the more biodegradable fibers are woven in two layers with the more biodegradable fibers on the exterior surface. In another embodiment, the structural fibers are woven (or laid down as a non-woven structure) and then the biodegradable material is applied as a coating to fill voids or reduce high spots in the sheath structure. In another embodiment, the structural fibers are woven (or laid down as a non-woven structure) with pliable appurtenances or with short, spiky or fur-like fibers protruding from the structural fiber mat. In this embodiment, a material that may transition from a moldable form to a more solid or gel form is applied in a manner which may force the appurtenances or short fibers to lay down on the surface and be held there as the biodegradable material transitions to a more solid or gel form.

The matrix of materials forming the sheath of a surgical suture may also comprise a porous woven, non-woven, or film-type construct, which may contain a medium within its porous matrix or on either its exterior surface, its interior surface, or both. The medium may be an emulsion, suspension, liquid and/or gel, which exhibits viscous, surface tension, or adhesive properties sufficient to be immobilized or retained therein, for a period of time. In one embodiment, the period of time may be sufficient to permit the suture described herein to be implanted at the site of the subject in need thereof. Additional time of immobilization may be employed to permit certain biological activities of the cells to occur within the confines of the suture prior to the cells and/or suture interacting with the host tissue—e.g., replication or differentiation of cells.

The medium embedded or impregnated may alternatively, or further include, at least in part, certain molecules having therapeutic value. These molecules may include antimicrobial, analgesic or anti-inflammatory molecules, while an underlying surface may contain other molecules or cells with therapeutic value including bioactive materials, such as stem cells. Antimicrobial molecules, at or near the surface, provide immediate functionality to address bacteria or viruses that may be introduced into the subject along with the suture. Bioactive molecules retained in an underlying surface may diffuse or otherwise be released over a longer time span, having an effect even after any infection has been addressed by the antimicrobials. The medium may include emulsion, suspension, liquid or gel, which may include materials with potential synergistic therapeutic benefits with the bioactive molecules. The medium may comprise oleic acid and/or linoleic acid. These molecules may have anti-microbial properties and benefits in concert with bioactive peptides, such as bone morphogenic protein.

In one embodiment, the surgical suture comprises an interior component comprising an exterior sheath, braided, woven or non-woven, including film, fibrous, or porous material and a porous interior core containing biological cells and/or biologically active molecules (collectively referred to herein as “bioactive materials”). The suture may optionally comprise a monofilament or multifilament polymeric material, or combinations thereof. The exterior sheath may optionally be porous and/or be provided, at least in part, with a hydrophobic material that may afford advantages of reduced wicking, both of polar liquids in the core to the outside of the suture, as well as fluids of the host tissue along the suture. One embodiment provides a porous sheath having a hydrophobic (also non-polar or less polar) liquid or gel introduced into its pores. This material may (i) serve as barrier for premature migration of cells and therapeutic material from the core of the suture to the exterior of the suture; (ii) reduce the infiltration of unwanted material into the suture core during handling; and/or (iii) reduce the friction of a porous suture as it is drawn through tissue and otherwise improve the handling characteristics of the suture.

In one embodiment, the suture may comprise a single-braided or woven construct as the interior core of the interior component. The interior core may be optionally covered by an exterior component in the interior component. The exterior component may comprises a barrier material. The barrier material may be a hydrophobic liquid or gel, but also may be a biodegradable solid, viscous film, or material having sufficiently high surface tension.

In another embodiment, in the surgical suture the exterior component is bound to the interior core material or to itself in a manner that constrains movement of the interior core relative to the exterior component along their longitudinal axes. This binding may be achieved by any suitable binding, depending on the application, including mechanical entanglement across the radius of the suture, thermal or chemical bonding of materials of the suture, or via a bonding material such as a staple.

Another embodiment provides an exterior sheath in the form of a hollow tube-like structure having inside and outside diameters, the sheath configured with constrictions at intervals along its length, such constrictions reducing the inside diameter to a point where it may reduce or preclude longitudinal migration or movement of material retained in the interior core of the suture. Such constrictions may be fanned before or after initial formation of the surgical suture. In one embodiment, the constrictions are introduced after the bioactive material of interest is introduced into the interior core. Constrictions may be formed by any suitable techniques, including mechanical deformation (e.g., crushing or twisting), thermal modification (e.g., melting), or introduction in the core of material in addition to the bioactive materials, where the additional material forms a blockage under some external stimulus. In one embodiment, the mechanical device that imparts a crushing or twisting action on a specific point in the suture construct may also be used to impart a mechanical pulling force on the suture itself, thereby providing the force needed to allow assembly of the suture.

In one embodiment, the exterior component (e.g., sheath) of the interior component of the suture provides at least a partial flow barrier, as well as protective benefits, and may assist in retaining cells and fluid media in the core during suture handling and implantation. As a result, the interior core may comprise materials which otherwise would not provide sufficient immobilization of biological cells and therapeutics for practical use in a suture. For example, multifilament cores may be used to entangle cells and/or therapeutics due to the restrictive flow paths around the filaments. Additionally, media comprising viscous liquids, foams, gels, and/or emulsions may also be utilized.

In one embodiment, biodegradable particles containing cells, bioactive molecules and other materials of therapeutic value may be employed to form the interior core of the suture, while a braided or woven structure of suture filaments may be employed to form the exterior sheath. The biodegradable particles may include polymer or natural constructs with embedded or surface expressed bioactive molecules, antimicrobial molecules, or other molecules of therapeutic interest. The particles may be of any size and geometric configuration, which is conducive to introducing the particles into the suture core, which permits a majority of particles to be retained in the core while the suture is implanted into the patient. The particles may have stem or other biological cells adhered to their surfaces or in a matrix, or they may have moieties that bind to the same.

Another embodiment provides nanowires, nanofibers, and/or microfibers, as binding or entanglement constructs in the interior core of a suture or as flow impeding constructs in a porous sheath. Further, such nanowires may exhibit a degree of entanglement in a multifilament suture structure, exhibiting benefits of retaining the biological cells. Additionally, nanowires may be induced to form hydrogels, which may be a part of the core of the suture.

In another embodiment, the porous interior component may comprise (a) a multifilament or a matrix of braided or woven filaments comprising the plurality of interstices disposed between the filaments; or (b) a porous monofilament comprising the plurality of pores, and wherein the biological cells are retained in the at least some of the plurality of pores or interstices. The pores or interstices herein may refer to discrete, or interconnecting pores or interstices, or a mixture of both.

In one embodiment, the suture may further comprise a bearing section and a contiguous leader section, wherein the bearing section comprises a length of a suture material comprising a therapeutically effective level of biological cells.

Suture with Biological Cells

The sutures described herein may contain biological cells or any of the aforedescribed bioactive molecules. In one embodiment, a suture is provided, the suture comprising: a porous interior component comprising a volume of pores or interstices, the volume comprising a binding surface; and a first number of biological cells dispersed in the volume. In one embodiment, the first number of biological cells in the volume is greater than a threshold number representing a second number of the biological cells otherwise at 100% confluence on the binding interior surfaces.

The suture, and the configuration and components thereof, may be any of those described. The volume may refer to a specific volume of the suture. The volume may refer to a portion of the suture or an entirety of the suture. The portion may be specified and designated in any suitable way.

The biological cells may be any of the biological cells described above. For example, the biological cells may comprise progenitor cells, stem cells, or a combination thereof. Other cells are also possible. The stem cells may be any of the aforedescribed stem cells, including, for example, mesenchymal stem cells. The cells may be obtained from any suitable sources, including those described above.

Depending on the cells involved, the cells may have any suitable size or geometry. In one embodiment described herein, the biological cells have a spherical (or approximately spherical) shape. An “approximately spherical” shape may refer to a spherical shape but with some minute amount of irregularity—but not enough to distract one of skill in the art from considering the shape as spherical. Some biological cells, including stem cells, including MSCs, may exhibit an (approximately) spherical shape when they are suspended in a medium. The medium may be any of those aforedescribed, including viscous liquid, foam, gel, emulsion, or combinations thereof. In some instances, a biological cell, such as a stem cell, may tend to change its morphology after it is cultured for a certain period of time. In the case of a stem cell, the stem cells tend to spread out after being cultured so that they become adherent to a surface. Thus, in one embodiment, the approximately spherical, or spherical, biological cells in the medium described herein may be those that have not yet undergone such transformation.

The term “confluency” in one embodiment herein refers to a measure of the number of the cells on a surface of a cell culture dish or a flask and refers to the coverage of the surface of the dish or the flask by adhered cells. The terms “adhered” and “adherence” in the context of cells herein refer to biological attachment of a cell to a surface in one embodiment. In one embodiment, in the context of a MSC, after the MSC becomes attached to a surface (i.e., become an “adhered” cell), the MSC tends to change its morphological configuration from a spherical (or approximately spherical) shape, as found in a suspension state, to a more flattened or spread out configuration on a surface. Accordingly, “100% confluence” of the cells in one embodiment may refer to a surface (of the dish) being about completely covered (100%) by adhered cells (i.e., there is no more room left for the cells to grow), whereas “50% confluence” refers to about half of the surface (of the dish) is covered (i.e., there is room for cells to grow). In one embodiment, the implant suture device may be designed to (i) minimize cell binding and resultant tissue growth (e.g., in cases where tissue growth might impair function of the implant); (ii) reduce immune response to the implant; or (iii) maximize cell binding, particularly in the case of scaffolds or other implants where incorporation into adjacent tissue is desired. It is generally believed as not preferable to overload a suture with cells because otherwise the cells would tend to die or other toxic effects might occur. In other words, one would normally want to have a cell number that is at 100% confluence or smaller. However, the present inventors discovered that for certain cells, overloading a suture with a high density of the cells will surprisingly have beneficial results.

The surface in one embodiment described herein may refer to a “binding surface,” which in one embodiment may refer to the surface area along the length of a section of suture intended for biological cell loading. The surface area may refer to an interior surface or an exterior surface of a suture. Depending on the context, the binding surface may refer to an interior surface of a suture. In another embodiment, the binding surface may refer to both the interior surfaces and the exterior surfaces along the length of a section of suture intended for biological cell loading.

An interior surface herein may refer to (any portion of) collectively the inner surfaces of the interstices or pores of the interior component (including the interior core and the exterior component), to which surface the cells may attach. An exterior surface may refer to (any portion of) the outermost surface of the suture exposed to the surrounding area; in the case of a suture with a sheath covering an inner core, the exterior surface may refer to the outermost surface of the suture, if there is no other component exterior relative to this component. The binding surface in a given configuration of the suture may be accessible to a biological cell such that a portion of the cell can contact the surface in a manner in which binding of the cell to the surface may be initiated; whereupon, under appropriate culture conditions, cell development to confluence may ensue.

A suture comprising only surfaces known to inhibit binding may have a very small effective binding area or even a binding area that is effectively zero. In this context, a suture with materials known or thought to prevent binding is still considered as having a binding surface, even if the effective area is taken as zero within the practical aspects of any calculation. Typical surface areas which would not represent potential binding surfaces (and thus not an area to the potential binding surface area) may refer to those in which there in no suture configuration where a portion of a biological cell of interest may physically contact the surface area in question—e.g., the interior surface of a pore of excessively small size or a completely enclosed pore. For example, activated carbon may have an extremely high surface area on a molecular scale. However, on a biological scale, the potential binding surface is significantly less because the majority of the surface may comprise pores too small to be accessible to biological cells. Nevertheless, even in this example, the larger exterior surfaces may still present an area that would be accessible to a biological cell. In one embodiment, a binding surface may refer to a surface of a material that may readily support binding and/or growth of the cells. In one embodiment, in the context of an implant, a binding surface may refer to an area of material to support binding and/or survival of biological cells for a period of time. The time period may be exceeding 12 hours—e.g., exceeding 24 hours, 36 hours, 38 hours, or more. In one embodiment, the binding surface may refer to the surface structure material of the implant, including a surface coating, which may promote or inhibit binding.

In one embodiment, the interior core and/or the exterior component comprise one or more surface molecules or films that represent potential binding or bonding sites for cells or therapeutic molecules. These molecules may be present anywhere at these components, such as at the binding surface thereof. The binding or bonding may arise through chemical conjugation, absorption, and/or hydrophobic interaction, or other mechanisms for bonding cells or molecules to substrate materials. In one embodiment, the biological cells, particularly the stem cells, bind to certain materials, most notably polymers (also plastics). Protein-coated polymers may also be employed due in part to their propensity to promote the binding and growth of cells.

In one embodiment, the cell density at confluence may depend at least in part on the binding nature of the material at the binding surface. The term “binding” herein may refer to chemical binding, physical binding, or a combination thereof, of the cells to a surface of the structure. In one embodiment, the binding may be used to describe how a cell is retained on the surface. The binding may refer to ionic binding, covalent binding, adsorption, absorption, entrapment, entanglement, or entrainment, or combinations thereof. In one embodiment, wherein the suture comprises silk coated with wax (as an exterior component), the cells at 100% confluence are very low. However, in another embodiment wherein the suture comprises silk not coated with wax, the number of cells approaches about 2000-3000 cells/cm².

The number of cells at 100% confluence on the binding surface may thus serve as an indicator of a threshold indicating whether the suture is overloaded with biological cells in a given volume. Depending on the cells involved and the material of the suture (and the nature of the binding surface), the threshold number may vary and may be any number. For example, the threshold number may be between about 100 and about 10,000 cells/cm²—e.g., between about 200 and about 9,000, about 500 and about 8,000, about 600 and about 7,000, about 800 and about 6,000, about 1,000 and about 4,000, about 2000 and about 3000, about 2200 and about 2,600 cells/cm².

The sutures need not be overloaded with biological cells in a given volume containing the pores and/or interstices. In some embodiments, the number of cells is such that the cells may be less than 100% confluence at the binding surface (in a given volume). For example, the number of cells may be that the cells are at smaller than or equal to about 90%—e.g., smaller than or equal to about 95%, about 90%, about 85%, about 80%, about 70%, about 60%, about 50%, or lower.

In some embodiments, the sutures described herein are overloaded with biological cells, such as stem cells, such as MSCs. In one embodiment, the sutures contain a first number (of the biological cells) being greater than at least about two times the threshold number of cells that would be considered to achieve 100% confluence—e.g., at least four times, ten times, 20 times, 50 times, 100 times, 200 times, or more. In one embodiment, the first number may be between about 2 times and about 1000 times—e.g., between about 4 times and about 800 times, between about 10 times and about 600 times, between about 20 times and about 600 times, between 40 and about 400 times, between about 60 and about 200 times, between about 80 times and about 100 times.

The number of the biological cells may be represented in various ways. For example, the number may be presented as being normalized by a dimension of the suture. The dimension may be any dimension, including length, width, diameter, etc. In one embodiment, the number is determined per length of the suture, and is greater than about 1 million cells per diameter of the suture—e.g., greater than about 2 million, 2.5 million, 3 million, 3.5 million, 4 million, 4.5 million, 5 million, 5.5 million, 6 million, 6.5 million, 7 million, 7.5 million, 8 million, 8.5 million, 9 million, 9.5 million, 10 million or more cells. Bigger or smaller numbers of the cells are also possible. In other words, the number may be represented by the expression N/L>1 million (cells)/D, wherein N, L, D is each a real number representing number of cells, a length, a diameter, of the suture.

Sutures with Different Levels of Hydrophobicity

The sutures described herein may have different levels of hydrophobicity among the different components of the sutures. One embodiment provides a suture, comprising: a porous interior component comprising pores or interstices; and a first number of biological cells dispersed in a plurality of the pores or interstices; wherein the suture has a plurality of portions having multiple levels of hydrophobicity. The sutures may have any of the configurations as described above.

In one embodiment, the hydrophobicity may also be described in terms of hydrophilicity, as a low hydrophobicity may refer to a high hydrophilicity, and vice versa. The different levels of hydrophobicity may be present in the suture in various ways. For example, the suture may comprise multiple sections lengthwise, and these sections may have different levels of hydrophobicity. For example, the hydrophobic section of the suture may be sandwiched between two hydrophilic sections, and vice versa. Alternatively, the suture may comprise alternating sections of hydrophobic section and hydrophilic section.

The levels of hydrophobilicity may also vary among components of the suture. In one embodiment, wherein the interior component comprises a porous interior core and an exterior sheath, at least a portion of the interior porous core is hydrophilic and at least a portion of the sheath is hydrophobic. The portion may be any size, depending on the application. In one embodiment, the entire interior porous core is hydrophilic and the entire sheath is hydrophobic. The hydrophobicity of the core and sheath may be reversed as described above. In one embodiment, the porous interior component comprises a braided structure of multiple filaments, at least some of the filaments being hydrophilic while at least some of the filaments are hydrophobic. In one embodiment, the suture has a sheath and core, and the sheath comprises a hydrophobic material and the core comprises, at least in part, a hydrophilic material.

One embodiment provides a porous surgical suture, in which exposed in the pores of the suture are hydrophilic (binding) surfaces. In one embodiment, a hydrophilic interaction with these surfaces may be a polar liquid medium comprising biological cells, such as stem or other progenitor cells. In one embodiment, the suture is overloaded with the biological cells—i.e., these cells are present in the pores of a given volume of the suture at a total number that exceeds the equivalent cell loading at 100% confluence on the binding surfaces in that given volume.

One embodiment provides that in a braided construct of a surgical suture with a high loading of biological cells (e.g., stem or other progenitor cells), the braid may comprise both fibers of a hydrophilic nature and fibers of a hydrophobic nature. This configuration may be employed to provide a suture with filaments of the braid that retain the cells in the pores through hydrophilic interaction while providing the suture with the enhanced tensile strength characteristics that are typical with a hydrophobic suture material.

One embodiment provides that the hydrophilic properties of the internal pores may also be achieved through introduction of hydrophilic material, either as a coating to the hydrophobic material filaments (either before or after braiding) or through the introduction of hydrophilic elements which are retained in the braid structure through entanglement or entrapment—e.g., with nanowires, particles, or hydrogels.

One embodiment provides that a suture may comprise different sections along its length where certain sections are of materials or are treated to be hydrophobic in nature and other sections are, at least in part, hydrophilic in nature. Further, the hydrophilic sections may also comprise a higher concentration of the biological cells, including stem cells and/or progenitor cells, than the adjacent hydrophobic section(s). In one embodiment, leader and/or tail sections of the length of suture may be hydrophobic in nature and a midsection of the length may be hydrophilic in nature. This configuration may be employed to concentrate the bioactive cells in a central part (also midsection) of the suture that will be positioned in the tissue, where the leader and/or tail sections may be employed for knot tying or facilitating handling during stitching.

Further, in one embodiment, the leader and/or tail sections of the length of suture may have additional properties that enhance the performance of the suture in strength, knot tying, and resistance to fraying, where those properties are not necessarily in common with the midsection of the length. Specifically, the leader and tail sections may have structures consistent with heat treatment typical of some sutures to reduce the tendency for the suture to fray where cut.

In certain applications, it is desirable to minimize or reduce the potential for wicking along the suture as it passes through a tissue boundary. In one embodiment, wicking occurs when fluid surface tension draws liquid along the length of a hydrophilic material from a region of high liquid content to a dryer area. Wicking may be a challenge in suture repairs that penetrate a tissue boundary, such as the skin, where a hydrophilic suture may result in weeping of the wound at the suture penetration points. One embodiment provides a suture in which a cell-loaded hydrophilic region may comprise alternating zones of hydrophobic and hydrophilic properties along the length of the suture. For example, the hydrophilic zones may comprise a higher loading of bioactive cells, and the length of any given hydrophilic zone may be limited so that the wicking potential across a tissue boundary of concern is reduced. In this case, the wicking potential may be established by the hydrophilic flow area on either side of the tissue boundary of a single hydrophilic zone.

In another embodiment, to restrict the potential for wicking, the hydrophilic section may be designed to be no longer than the thickness of the tissue boundary of concern. For example, human skin (at least in some instances), is about 2 mm thick and thus if the hydrophilic zone between two hydrophobic zones of a suture is no more than about 2 mm long (e.g., 1 mm, 0.5 mm, or smaller), the potential for hydraulically bridging across the tissue would be greatly reduced. Other dimensions may be used as well, depending on the tissue and suture involved. In one embodiment, a size 4-0 surgical suture is at most about 2 mm in diameter, and thus a series of 2 mm diameter×2 mm long hydrophilic zones in the active part of a suture may be employed. In one embodiment, the intervening hydrophobic zones need to be only of sufficient length to minimize (or even prevent) hydraulic breakthrough between hydrophilic zones. This is dependent at least in part on the stability of the hydrophobic zone and the ability to manufacture the hydrophobic zone.

In one embodiment, the difference between adjacent hydrophilic and hydrophobic zones may be produced after the basic suture construct is formed (e.g., braided). The specific zones may be coated or treated to impart hydrophobic or hydrophilic properties, depending on whether the underlying construct is hydrophilic or hydrophobic, respectively. For example, a hydrophilic section of suture may be broken up into zones by coating certain zones with a wax or other hydrophobic material. In another embodiment, these treatments to induce hydrophilic or hydrophobic properties may include a visual indicator, such as shade or color, to provide an indication of which zones in the suture may contain or be made to contain bioactive cells. Further, one embodiment provides that the medium which contains bioactive cells may also comprise a dye or other indicator, which assists in identifying which zones may contain bioactive cells or even whether a suture has been loaded with bioactive cells.

### Implantation

Provided in some embodiments are methods of using the sutures described herein. The use may involve implantation into a subject in need thereof In one embodiment, the sutures described may be employed utilizing methods to reduce or retard temporarily the cellular activities within a period of time from loading the suture with cells. The period may be of any length, depending on the application. For example, the period may be at least about 6 hours, at least about 8 hours, at least about 10 hours, at least about 12 hours, at least about 14 hours, or more.

One embodiment described herein provides a method of implanting a suture into a subject in need thereof. The method comprises: exposing a suture to a medium comprising a concentration of biological cells, such that a first number of the biological cells are dispersed in the volume of the pores or interstices; and implanting the suture into the subject. The suture may be any of the sutures described herein. The medium may be any of the media described herein.

The step of exposing during the methods of implantation described herein may be carried out for any suitable time, depending on the cells, materials etc., involved. In one embodiment wherein the cells have at least approximately spherical configuration, the step of exposing is carried out sufficiently rapidly that at least some of the biological cells will have not had a chance to change their morphology and thus will retain at least an approximately spherical configuration. In one embodiment, the time is such that at least substantially all, such as completely all, of the biological cells retain at least an approximately spherical shape.

Not to be bound by any particular theory, but because the suture is overloaded with biological cells (stem cells, such as MSCs, in one embodiment), the suture may deliver a larger number of cells to the tissue site than a suture otherwise not loaded with as many cells. The cells may facilitate tissue repair (including regeneration), and thus have a large number of such cells that may improve tissue repair. Such an approach contradicts the conventional belief that concentrations of cells above what might be achieved in a cell culture should be avoided because of the natural tendency of biological systems to fail when loaded to excess; this has been shown in the past particularly in the case of mesenchymal stem cells, which trigger apoptosis (also programmed death) in a population when the concentration is too high. The new approach is reported for the first time by the present inventors herein.

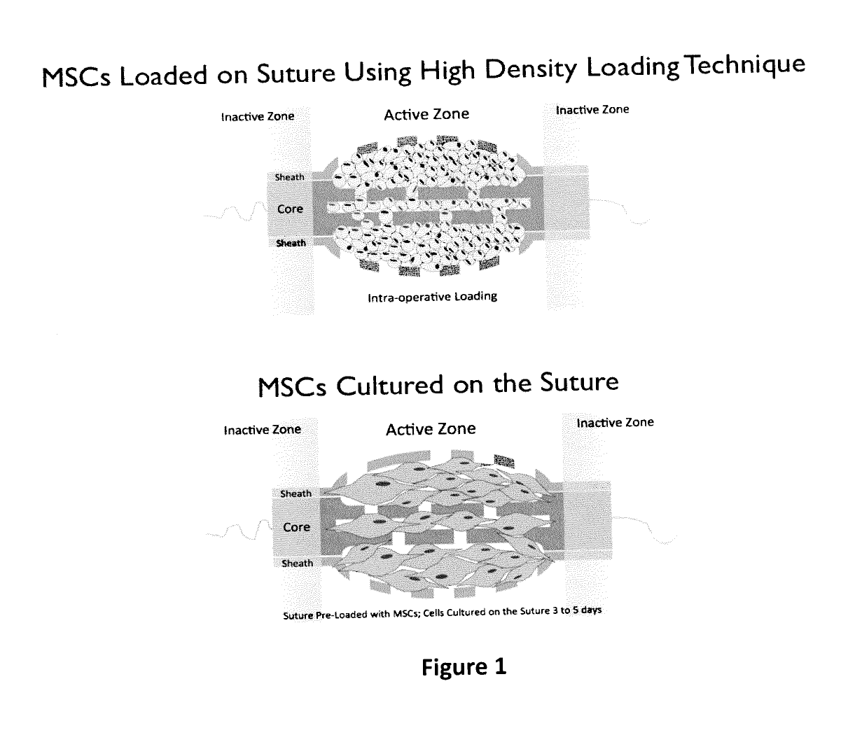

The stem cell bearing sutures described herein are loaded with densely concentrated MSCs in their unattached (spherical) form. When the cells are spherical, it is possible to compact them very tightly in a small space; when the MSCs are adherent they have a much larger surface area than when they are in their spherical form. Not to be bound by any particular theory, but due to their larger surface area, fewer adherent MSCs may inhabit the suture than unattached (spherical) MSCs because there is physically not enough room for adherent MSCs to fit on the suture at such high densities (see FIG. 1). When the suture is loaded with very high numbers of densely concentrated MSCs and attempts are made to culture those cells on the suture, the MSCs start to spread out to attach. Inevitably some of the MSCs will die from lack of space or will be pushed out of the suture and into the in vitro environment and therefore never make it to the site of repair.

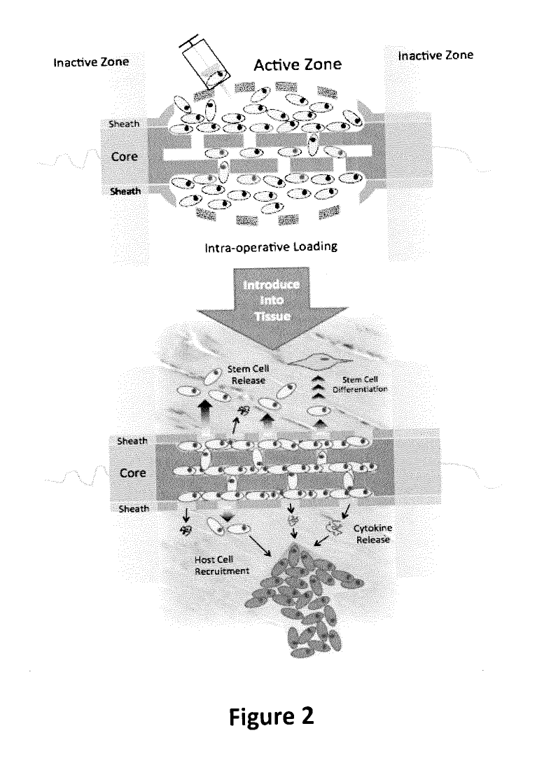

On the other hand, when the high density stem cell loading method is used, the MSCs may occupy the pores or interstices within the interior core of the suture or, in a multilayer suture construction, in any space between layers. In one embodiment, immediately after loading cells in the suture, the suture is introduced to the site of repair in the patient subject. Once implanted, at least some of the MSCs may migrate out of the interstices of the suture and into the surrounding pathological tissue (see FIG. 2). Implanted MSCs may undergo differentiation and secrete cytokines to aid in tissue healing and recruitment of host cells. By implanting the suture in a relatively short time period after loading the cells into the suture, cell-density-based apoptosis may be reduced (or even minimized) before the suture is implanted to an environment (e.g., target tissue of the subject) where the cells may begin to migrate out of the suture.

In other embodiments, the cells may be loaded into the suture well before implantation, but steps may be taken to reduce biological (e.g., cellular) activity in order to forestall undesired cell-density-based apoptosis. These steps may include introduction into a low temperature environment (e.g., freezing), chemical treatments, or a combination thereof.

### Kit

One embodiment provides the inclusion of one or more surgical suture needles or analogous devices in the packaged kit for use in conjunction with the suture material. The needle may facilitate the penetration of tissue and pulling of the suture material through the tissue, and, thus may be integrated with or attached to the suture material while in the package to alleviate a need for threading the suture material into a needle or attaching it thereto. In one embodiment, the sharp tip of the needle may be provided with a point protector—e.g., a piece of material that the point cannot easily puncture. Alternatively, the needle may be mounted in the package in a manner that reduces potential puncture wounds during handling or opening of the package.

The packaged kit may include suture material in any configuration that allows for the dispensing of the suture material in a sequential manner without tangling. Such dispensing systems may be similar to those conventionally used for dispensing suture material, string, wire, dental floss and tape, thread, and similar material. In one embodiment, the package may contain a length of suture material suitable for use in a single patient, or in a single wound. Alternatively, the package may contain suture material in multiple discrete lengths or a continuous length that may be separated into discrete lengths.

Kits and packages described herein may include an area within or outside of the sterile package where materials extracted from a prospective patient, for example those presumed to include stem cells, other biological cells of interest and/or molecules of therapeutic value (bioactive materials), may be introduced as part of a medium—e.g., liquid solution and/or bioactive solution. The package design may be such that the suture material is in physical contact with this medium while it is in the package or as it is extracted from the package for use. One embodiment provides that the area of contact between said bioactive solution and the suture material may take the form of a separate or isolated zone, such as a sterile zone within the package, that is designed to accept the medium introduced through a hypodermic needle or analogous dispensing device into a zone which is configured to maximize the contact between solution and suture material (while in the package) or as the suture material is extracted from the package. Such a configuration may take the form of an expandable area containing the suture material, such that the suture material is in immediate contact with the solution when the solution is introduced into the package. Such a configuration may also involve a tube-like or other geometry section that holds the medium and which the suture must pass through as it is drawn from the package. This configuration may be employed to minimize the potential of bioactive molecule containing fluid sitting in unproductive pockets, which are not disturbed by the passing of the suture material through said area of the package. More complex mechanisms may also be employed for contacting bioactive molecule containing fluid with the suture material.

One embodiment provides that the surgical suture or precursor construct contained within the sterile package may include a leader section of suture material. The leader section is a length of suture material which is not intended to include bioactive materials but is contiguous with the remainder of the suture material which does include bioactive materials. The leader section may extend from a zone where suture material and bioactive molecule solution are in contact with an area outside that zone, wherein the leader section may be accessed to be grasped, by hand or tool, and pulled to extract the bioactive molecule coated section of the suture from the contacting zone.

Further, the leader section may optionally be treated so as to provide for more effective termination of the suture in a wound repair, such as with material that exhibits improved knot holding characteristics. A tail (or distal) end of the suture may exist on the opposite side of the bioactive materials zone of the suture, where said tail end possesses similar properties as the leader section. The leader portion, and optionally tail portion, of the suture material may be differentiated by (a) a visual identifier such as a difference in color, shade, texture and pattern, and/or (b) a difference in one or more physical properties such as concentration of bioactive material, knot holding characteristics and handling characteristics. Accordingly, the suture material may be positioned in the package such that the suture material forms a continuous chain from the zone where the bulk of the suture material is stored, for example, through the zone where the bioactive material may be added, which may or may not be the same zone where the bulk of the suture material is stored, into the leader zone of the package where optionally a needle or other such device may be packaged, and where the package may be conveniently opened by the user at the time of use. Consequently, in one embodiment the package may be opened by some mechanisms of access to the leader zone, which access may be cutting or tearing open the packaging at the leader zone to expose the suture leader material or surgical needle.

A surgical suture may be hydraulically connected to the interior of a dispensing syringe (also hypodermic) needle, tube or other analogous device which is, in turn, attached to a reservoir which may contain bioactive material or, in the kit in one embodiment, is connected to the package zone where bioactive materials are introduced. In one embodiment, bioactive materials may be injected directly into the interior core of the suture. In another embodiment, the bioactive materials may be introduced into the pores and interstices in the interior core of the suture by inducing flow of material through a porous suture exterior and into the interior. Consequently, flow of bioactive material into the interior pores or interstices of a suture can be either induced by fluid flow along the longitudinal axis of the suture, inward along a radius of the suture, or both.

In one embodiment, a high concentration of stem cells to be introduced into a suture may be obtained by concentrating cells with a centrifuge to a high concentration—e.g., to the point of pellet formation. The excess fluid is removed and optionally a different medium or a known volume of the same medium is reintroduced, and the concentrated cells are agitated to disperse them in a more uniform manner at the desired concentration.

### NON-LIMITING WORKING EXAMPLES

### Example 1

Human MSCs were obtained from bone marrow aspirate using conventional techniques. The MSCs were then grown to 90% confluence on a tissue culture flask in an incubator at 37° C. with 5% CO₂ over a one week period. The MSCs were rinsed with 3 mL PBS, and then the excess PBS was aspirated from the culture flask. 1.5 mL Trypsin-EDTA was added to the MSCs in the tissue culture flask and the flask was incubated at 37° C. for 5 minutes. The Trypsin-EDTA was then neutralized with 3 mL Mesenchymal Stem Cell Basal Medium. The solution was transferred to a 15 mL conical tube and spun down in the centrifuge at 2500 rpm for 10 minutes. The supernatant was aspirated off, and the cell bearing pellet was then re-suspended in 1 mL Mesenchymal Stem Cell Basal Medium. The MSCs were then counted using a hemacytometer. A solution medium containing approximately 2 million MSCs were then spun down in the centrifuge at 2500 rpm for 10 minutes, the supernatant aspirated off and the cell bearing pellet was re-suspended in 15 μL of Mesenchymal Stem Cell Basal Medium to yield the medium for loading into a suture.

The suture was formed by braiding 20 denier silk in a 2 ply braid at 81 PPI over an 18 gauge PTFE core. The braided silk was removed from the core material. A 20 cm length of this material was drawn into the center of a 15 cm length of the same material to form a sheath and core silk suture of a No. 2 size suture. Leader and tail sections of this suture construct were treated with a medical grade wax, leaving a 2.54 cm section of uncoated silk suture in the center. The suture was drawn into a 4 cm section of 18 gauge PTFE tubing; the tubing was then positioned over the uncoated silk portion of the suture. The 15 μL of MSC-bearing solution was injected in to the PTFE tube such that it saturated the interior of the section of uncoated silk suture, whereupon the PTFE tube was removed from the suture. No residual MSC-bearing medium was observed in the tube. The bioactive cell bearing section of the suture was then tested to determine the actual live cell loading (i.e., cell content). 1.4 million cells were found to be contained within the 2.54 cm section suture.

### Example 2

A No. 2 stem cell bearing suture was constructed as described in Example 1, with the exception that cell loading was at 1 million cells in the medium with an actual retention of 770,000 cells on average in the suture. The suture was then used to repair a 3 mm gap incised in the hind quarter tendon of a laboratory rat under appropriate laboratory protocols. A 3 mm gap in a second hind quarter tendon was repaired using a suture without MSCs, as a negative control. At day 28, the repaired tendons were compared. The tendon repaired with the suture loaded with MSCs were found to have developed stronger and more orderly tissue (less scar tissue). Specifically, Example 2 is described in more detail below:

An animal study was performed to evaluate the efficacy of the No. 2 stem cell bearing suture to contribute to tendon healing properties in a rat Achilles tendon repair model.

Human MSCs used in the study were obtained from bone marrow aspirate from one human donor under Institutional Review Board approval. A 3 mm trocar with obturator needle were used to aspirate bone marrow from either the calcaneus, proximal tibia, and/or the iliac crest. Bone marrow aspirate (“BMA”) was mixed with citrate-based anticoagulant (13-17% v/v) to prevent coagulation. 55±14 mL of anticoagulated BMA was collected from each patient. BMA was processed using the MarrowStim™ Concentration System (Biomet Biologics, LLC, Warsaw, Ind.). Following 15-minute centrifugation at 1400×g, 3-6 mL of aspirate cell concentrate (“ACC”) was transferred to the lab for MSC isolation, culture, and expansion.

The sample was processed to remove the red blood cells and to isolate mononuclear cells. This processing was achieved using Ficoll-Paque™ PLUS (STEMCELL Technologies, Vancouver, Canada) which is a density gradient medium for isolation of mononuclear cells. The fresh bone marrow aspirate was diluted at a 1:1 ratio with phosphate buffered saline (“PBS”) pH 7.4 (Sigma-Aldrich, St. Louis, Mo.) with 2% fetal bovine serum (“FBS”) for human mesenchymal stem cells (STEMCELL Technologies, Vancouver, Canada). The diluted bone marrow aspirate was then layered on top of the Ficoll-Paque™ PLUS density gradient medium in a 50 mL conical tube and centrifuged at room temperature for 30 minutes at 400×g with the centrifuge brake off. The layer containing MSC mononuclear cells at the plasma-Ficoll interface was then removed and the mononuclear cells were washed once with PSB containing 2% FBS before being plated in new T-75 cm² tissue culture flasks at a density of 4000 cells per cm². The isolated MSCs were cultured in MesenCult® MSC Basal Medium (STEMCELL Technologies, Vancouver, Canada) supplemented with MesenCult® Mesenchymal Stem Cell Stimulatory Supplements (STEMCELL Technologies, Vancouver, Canada). The cells were expanded in a 37° C. humidified incubator with 5% CO₂ in air and >95% humidity for 14 days or until they were 60-80% confluent, at which point the adherent cell monolayer was dissociated from the cell culture flask using 0.25% Trypsin-EDTA (GIBCO® Invitrogen, Cat #15050-057). The dissociated MSCs were centrifuged for 10 minutes at 500×g, counted, and re-plated in new T-75 cm² tissue culture flasks at a density of 4000 cells per cm².

The MSCs were expanded with MesenCult® MSC Basal Medium supplemented with MesenCult® Mesenchymal Stem Cell Stimulatory Supplements and kept in a tissue culture incubator at 37° C. with 5% CO, in air and >95% humidity. The medium was replaced every 3 days until the MSCs were 60-80% confluent, at which point they were dissociated from the cell culture flask using 0.25% Trypsin-EDTA, centrifuged for 10 minutes at 500×g, and counted. 1×10⁶ of the passage two mesenchymal stem cells concentrated in 15 μL were impregnated per inch of the stem cell suture, with an average of 770,000 MSCs retained per inch of the suture and delivered to the repair site.

Three different suture repair types were tested in this study—a repair using only suture (Suture Only), a repair using suture plus the injection of 1×10⁶ stem cells around the repaired tendon (Suture+SC Injection), or a repair using a suture impregnated with 1×10⁶ stem cells (SC Suture). All sutures used were constructed by braiding 20 denier silk in a 2 ply braid at 81 PPI over an 18 gauge PTFE core. The braided silk was removed from the core material. A 20 cm length of this material was drawn into the center of a 15 cm length of the same material to form a sheath and core silk suture of a No. 2 size suture. Leader and tail sections of this suture construct were treated with a medical grade wax, leaving a 2.54 cm section of uncoated silk suture in the center. The suture was drawn into a 4 cm section of 18 gauge PTFE tubing; the tubing was then positioned over the uncoated silk portion of the suture. For the stem cell suture repair type, 15 μL of medium containing 1×10⁶ MSCs (as described above) was injected in to the PTFE tube such that it saturated the interior of the section of uncoated silk suture. The PTFE tube was removed from the suture. No residual MSC-bearing medium was observed in the tube. For the suture only and the suture plus stem cell injection repair types, the PTFE tube was removed from the suture immediately before use in the animal; no changes were made to the section of uncoated silk suture.

Prior to the start of the procedure, each hind-limb was randomized to receive one of the three repair types. Fifty-four adult male Sprague-Dawley rats with a mean weight of 370 gm (ranging from 326-600) were anesthetized prior to incision by intraperitoneal injection of Nembutal (pentobarbital); bilateral hind-limbs were then shaved and prepped with alternating alcohol and chlorhexidine scrubs three times. A longitudinal posterior midline incision was made along the Achilles tendon to expose the Achilles tendon. Once identified and isolated, the Achilles tendon was transected 3 mm distal to the musculotendinous junction. A subsequent second transection was performed 3 mm distal to the first, resulting in the removal of a 3 mm section of the tendon. The Achilles tendon ends were loosely approximated, using the appropriate suture repair type, through a figure-of-eight stitch with three knots. Prior to tying the knots, an instrument 3 mm in width was placed between the tendon ends to preserve the 3 mm gap. For all the rats the wounds were irrigated, and the skin was closed with 4-0 monofilament suture in a subcuticular fashion.

Histological and biomechanical testing was performed at 14 and 28 days after repair. Biomechanical strength (load to failure) was the primary result of the repair during testing, while histological analysis was a secondary result. An a priori power analysis, based on biomechanical data from prior reports of rat tendon healing, determined that 12 tendons were needed per treatment group per time period to establish 90% confidence with a Type-1 error of 5%. An additional six tendons per treatment group per time period were used for histological evaluation. All histological and biomechanical results were expressed as means and standard deviations. As the Board Certified Pathologist was a single reviewer, 20 slides were chosen at random and blindly graded twice. A Cohen's Kappa value for intra-rater reliability was calculated. Biomechanical data at each time-point were analyzed across the three treatment groups using one-way ANOVA with Tukey's post hoc analysis. Biomechanical data from each treatment group were also compared as a function of time-point (of measurements) using a two-tailed t-test. Histological ordinal data at each time point were analyzed across the three treatment groups using a Kruskal-Wallis test with Bonferonni post-hoc correction. Histological data from each treatment group were compared between each time point using chi-square analysis. Statistical significance was set at p<0.05 except for Bonferonni correction in which significance was p<0.016. Statistical analysis was performed using JMP 9.0.0 statistical software (SAS Institute, Inc., Cary, N.C.).

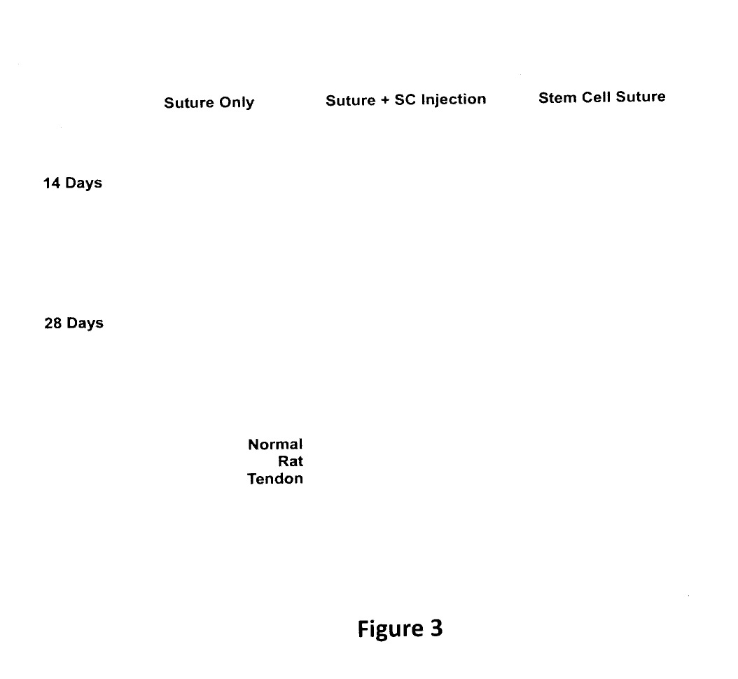

The biomechanical and histological results are shown in FIG. 3. FIG. 3 demonstrated that the tendon repaired with the suture loaded with a large number MSCs had developed stronger and more orderly tendon tissue (less scar tissue). At day 14, the tendons repaired using the stem cell suture were statistically healthier and displayed more native-like tendon than either of the other repair types. At day 28, the tendons repaired using the suture with stem cell were statistically healthier and displayed more native-like tendon than the Suture Only suture (Table 1). The grading scheme as shown in Table 1 is in accordance with that provided in Rosenbaum et al, Histologic Stages of Healing Correlate with Restoration of Tensile Strength in a Model of Experimental Tendon Repair, HSSJ (2010) 6:164-170. That is, the values in Table 1 are on a scale, where 0 represents a healthy tendon and 3 represents a diseased tendon. Biomechanical results revealed that at both time points the tendons repaired using the stem cell suture repair method had a statistically significant higher peak stress than the tendons repaired using either of the other repair types (Table 2).

TABLE 1
Mean Biomechanical Grades for Each Tendon Repair Type

| Time | | Suture | Suture + | SC |
|---|---|---|---|---|
| Point | Component | Only | SC Injection | Suture |
| 14 Days | Collagen Grade | 1.9 ± 0.8 | 1.6 ± 0.9 | 0.4 ± 0.3 |
| | Angiogenesis | 0.8 ± 0.2 | 0.9 ± 0.3 | 1 ± 0 |
| | Cartilage Formation | 0 | 0 | 0 |
| | Total Score | 2.7 ± 0.8 | 2.5 ± 0.8 | 1.4 ± 0.3* |
| 28 Days | Collagen Grade | 2.4 ± 0.6 | 2.1 ± 0.7 | 1.1 ± 0.2 |
| | Angiogenesis | 1 ± 0 | 0.7 ± 0.4 | 0.9 ± 0.3 |
| | Cartilage Formation | 0 | 0.1 ± 0.1 | 0 |
| | Total Score | 3.4 ± 0.6 | 2.8 ± 0.8 | 2.1 ± 0.3†‡ |

SC = Stem Cell
* = statistically different from Suture Only and Suture + SC Injection groups
† = statistically different from Suture Only group
‡ = statistically different from the 14 day time point

TABLE 2
Biomechanical Properties of Each Tendon Repair Type

| Time | | Suture | Suture + | SC |
|---|---|---|---|---|
| Point | Property | Only | SC Injection | Suture |
| 14 Days | Ultimate Failure Load (N) | 22.3 ± 5.3 | 22.3 ± 7.0 | 19.0 ± 5.9 |
| | Area (mm²) | 18.8 ± 5.1 | 9.7 ± 5.0† | 8.2 ± 4.8† |
| | Peak Stress (Mpa) | 1.2 ± 1.0 | 2.3 ± 1.4 | 2.3 ± 1.2† |
| 28 Days | Ultimate Failure Load (N) | 21.8 ± 5.4 | 20.7 ± 6.8 | 18.8 ± 4.3 |
| | Area (mm²) | 18.3 ± 3.9 | 14.3 ± 5.8‡ | 9.0 ± 2.8* |
| | Peak Stress (Mpa) | 1.2 ± 0.4 | 1.6 ± 0.9‡ | 2.3 ± 0.8† |

SC = Stem Cell
† = statistically different from Suture Only group
* = statistically different from Suture Only and Suture + SC Injection groups
‡ = statistically different from the 14 day time point

### Example 3

An in Vitro study was conducted to investigate the efficacy of a No. 2 stem cell bearing suture constructed using the high density loading technique in one of the embodiments described above to deliver MSCs to a site of surgical repair. Three different stem cell culturing time points for MSCs loaded on No. 2 stem cell bearing sutures were tested in this study: (1) No. 2 stem cell bearing suture 30 cm in length loaded with an average of 33,333 MSCs per cm, stem cells cultured on the suture for 3 days; (2) No. 2 stem cell bearing suture 18 cm in length loaded with an average of 55,555 MSCs per cm, stem cells cultured on the suture for 5 days; (3) No. 2 stem cell bearing suture 18 cm in length loaded with an average of 535,871 MSCs per cm, high density stem cell bearing suture used immediately at the site of surgical repair.

The No. 2 stem cell bearing sutures were constructed using the same protocol as that described above. Human MSCs used in the study were obtained from bone marrow aspirate from one human donor under Institutional Review Board approval using the techniques described in Example 2. The isolated MSCs were cultured in MesenCult® MSC Basal Medium (STEMCELL Technologies, Vancouver, Canada) supplemented with MesenCult® Mesenchymal Stem Cell Stimulatory Supplements (STEMCELL Technologies, Vancouver, Canada). The cells were expanded in a 37° C. humidified incubator with 5% CO₂ in air and >95% humidity for 14 days or until they were 60-80% confluent at which point the adherent cell monolayer was dissociated from the cell culture flask using 0.25% Trypsin-EDTA (GIBCO® Invitrogen, Cat #15050-057). The dissociated MSCs were centrifuged for 10 minutes at 500×g, counted, and re-plated in new T-75 cm² tissue culture flasks at a density of 4000 cells per cm².

The MSCs were expanded with MesenCult® MSC Basal Medium supplemented with MesenCult® Mesenchymal Stem Cell Stimulatory Supplements and kept in a tissue culture incubator at 37° C. with 5% CO₂ in air and >95% humidity, media was replaced every 3 days until the MSCs were 60-80% confluent at which point they were dissociated from the cell culture flask using 0.25% Trypsin-EDTA, centrifuged for 10 minutes at 500×g, and counted.

At the 3-day culturing time point, 1×10⁶ of the passage two MSCs were concentrated in 60 μL of Mesenchymal Stem Cell Basal Medium to yield the medium for loading into the suture. 33,333 MSCs in 2 μL were impregnated per inch of the stem cell suture. The stem cell impregnated suture was then placed in a sterile 100 mm tissue culture dish with MesenCult® Mesenchymal Stem Cell Basal Medium (STEMCELL Technologies, Vancouver, Canada) supplemented with MesenCult® Mesenchymal Stem Cell Stimulatory Supplements (STEMCELL Technologies, Vancouver, Canada). The MSCs were cultured on the suture in a 37° C. humidified incubator with 5% CO₂ in air and >95% humidity for 3 days. At day 3, the stem cell impregnated suture was removed from the incubator and assembled as it would be for surgery. A 2 mL plastic eppendorf tube filled with 500 μL PBS was used to simulate a site of surgical repair. The entire length of the stem cell bearing suture was placed in the eppendorf, and the number of stem cells delivered to the PBS and remaining on the suture were determined using luminescence to test for live cells. This was deemed a viable model because in a surgical setting the stem cell bearing suture would be assembled for surgery and then implanted immediately at the surgical site. Viable stem cells on the suture would either be delivered to the surrounding tissue at the site of repair or remain on the suture.

At the 5-day culturing time point, 1×10⁶ of the passage two MSCs were concentrated in 25 μL of Mesenchymal Stem Cell Basal Medium to yield the medium for loading into the suture. 55,555 MSCs in an average of 1.39 μL were impregnated per inch of the stem cell suture. The stem cell impregnated suture was then placed in a sterile 100 mm tissue culture dish with MesenCult® Mesenchymal Stem Cell Basal Medium (STEMCELL Technologies, Vancouver, Canada) supplemented with MesenCult® Mesenchymal Stem Cell Stimulatory Supplements (STEMCELL Technologies, Vancouver, Canada). The MSCs were cultured on the suture in a 37° C. humidified incubator with 5% CO₂ in air and >95% humidity for 5 days, with the medium changed after 3 days. At day 5, the stem cell impregnated suture was removed from the incubator and assembled as it would be for surgery. A 2 mL plastic eppendorf tube filled with 500 μL PBS was used to simulate a site of surgical repair. The entire length of the stem cell bearing suture was placed in the eppendorf and the number of stem cells delivered to the PBS and remaining on the suture were determined using luminescence to test for live cells.

For the high density loaded stem cell bearing suture used immediately at the site of surgical repair, an average of 1,444,444 passage two MSCs were concentrated in 12.7 μL of Mesenchymal Stem Cell Basal Medium to yield the medium for loading into the suture. An average of 535,871 MSCs in 0.7 μL were impregnated per inch of the very densely loaded stem cell suture. The stem cell impregnated suture was immediately assembled as it would be for surgery. A 2 mL plastic eppendorf tube filled with 500 μL PBS was used to simulate a site of surgical repair. The entire length of the stem cell bearing suture was placed in the eppendorf and the number of stem cells delivered to the PBS and remaining on the suture were determined using luminescence to test for live cells.

The number of live cells on the sutures was determined using the Cell Titer Glo Luminescent Cell Viability Assay (Promega). 500 μL of Cell-Titer Glo was added to each eppendorf containing the stem cell bearing suture and 500 μl PBS. The contents of the eppendorf were shielded from light, mixed for 2 minutes and then incubated at room temperature for 10 minutes. Following the 10 minute incubation, the number of viable cells on the suture and at the simulated repair site was determined using luminescence. Luminescence was recorded using the GloMax Multi Jr with luminescence module (Promega).

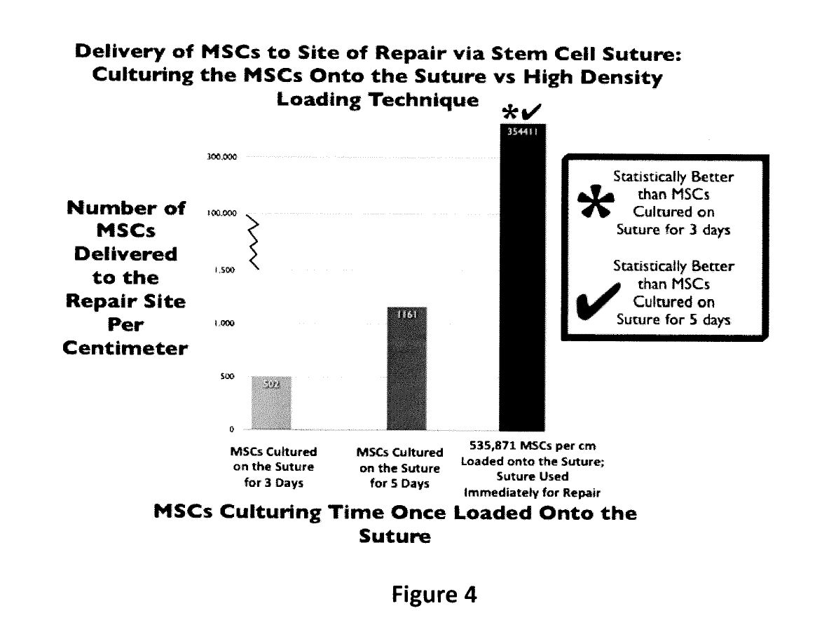

The results are shown in FIG. 4. As shown in FIG. 4, the high density loaded stem cell bearing suture used immediately at the site of surgical repair had an average of 354,411 MSCs per cm of suture delivered to the site of surgical repair. The stem cell bearing sutures with MSCs cultured on them had averages of 502 MSCs per cm of suture delivered to the site of surgical repair when the stem cells were cultured on the suture for 3 days and 1,161 MSCs per cm of suture delivered to the site of surgical repair when the stem cells were cultured on the suture for 5 days. The data was analyzed across the three stem cell bearing suture groups using one-way ANOVA with Tukey's post hoc analysis. Statistical analysis revealed that significantly more MSCs were delivered to the site of repair when the high density stem cell loading method was used than when MSCs were directly cultured on the suture for 3 or 5 days.

When the MSCs were loaded onto the sutures, they were in a medium solution in their unattached (spherical) form. At the 3- and 5-day culturing time points, 33,333 and 55,555 MSCs per cm were loaded onto the suture and then those MSCs were cultured directly on the suture for 72 and 120 hours, respectively. Culturing the MSCs onto the suture allowed them to attach to the suture fibers and to undergo proliferation. When MSCs took on their adherent form, they were found to elongate and spread out. As described above, and based on the results herein, the number of cells ultimately delivered to the tissue of the subject is lower than that in the suture overloaded with the MSCs.

### Conclusion

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, the technology described herein may be embodied as a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” Any ranges cited herein are inclusive.

The terms “substantially” and “about” used throughout this Specification are used to describe and account for small fluctuations. For example, they may refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of shall be closed or semi-closed transitional phrases, respectively, as set forth in the United States Patent Office Manual of Patent Examining Procedures, Section 2111.03.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed:

1. A suture, comprising:
a porous interior component comprising a volume of pores or interstices, the volume comprising a binding surface; and
a first number of biological cells dispersed in the volume;
wherein the first number of biological cells in the volume is greater than a threshold number representing a second number of the biological cells at 100% confluence on the binding surface.

2. The suture of claim 1, wherein the porous interior component comprises:
a porous interior core; and
an exterior component exterior relative to the interior porous core.

3. The suture of claim 1, wherein the first number is greater than at least about two times the threshold number.

4. The suture of claim 1, wherein the first number is greater than at least about four times the threshold number.

5. The suture of claim 1, wherein the first number is greater than at least about ten times the threshold number.

6. The suture of claim 1, wherein the threshold number is between 2000 and 3000 cells/cm².

7. The suture of claim 1, wherein the first number is determined per length of the suture and is greater than about 7,500,000 cells per diameter of the suture.

8. The suture of claim 1, wherein the biological cells are retained in the pores or interstices by ionic binding, covalent binding, adsorption, absorption, entrapment, entanglement, or entrainment, or combinations thereof.

9. The suture of claim 1, wherein the biological cells comprise progenitor cells or stem cells that have an approximately spherical configuration.

10. The suture of claim 1, wherein the biological cells in the volume of the pores and interstices are in a medium that is at least one of viscous liquid, foam, gel, and emulsion.

11. The suture of claim 1, wherein the biological cells comprise mesenchymal stem cells.

12. The suture of claim 1, wherein the porous interior component further comprises:
(a) a multifilament or a matrix of braided or woven filaments comprising the plurality of interstices disposed between the filaments; or
(b) a porous monofilament comprising the plurality of pores, and
wherein the biological cells are retained in the at least some of the plurality of pores or interstices.

13. The suture of claim 1, further comprising a bearing section and a contiguous leader section, wherein the bearing section comprises a length of a suture material comprising a therapeutically effective level of biological cells.

14. The suture of claim 1, comprising multiple sections along a length of the suture, the multiple sections having multiple levels of hydrophobicity.

15. The suture of claim 1, wherein the porous interior component comprises:
a porous interior core; and
an exterior component exterior relative to the interior porous core,
wherein at least a portion of the porous interior porous core is hydrophilic and at least a portion of the exterior component is hydrophobic.

16. A method of implanting a suture to a subject in need thereof, comprising:
exposing a suture, which comprises a porous interior component comprising a volume of pores or interstices, the volume comprising a binding surface, to a medium comprising a concentration of biological cells, such that a first number of the biological cells are dispersed in the volume of the pores or interstices; and
implanting the suture into the subject;
wherein the concentration of the biological cells is sufficiently high such that the first number of the biological cells is greater than a threshold number representing a second number of the biological cells at 100% confluence on the binding surface.

17. The method of claim 16, wherein the threshold number is between 2000 and 3000 cells/cm².

18. The method claim 16, wherein the exposing is carried out sufficiently fast such that at least some of the biological cells retain an approximately spherical configuration.

19. The method of claim 16, wherein the implanting results in a larger number of the biological cells being transferred to the subject than a different suture having biological cells cultured on the suture before implantation.

20. The method of claim 16, further retarding cellular activities of the biological cells for at least about 8 hours after the exposing.

21. A suture, comprising:
a porous interior component comprising pores or interstices; and
a first number of biological cells dispersed in a plurality of the pores or interstices;
wherein the suture has a plurality of portions having multiple levels of hydrophobicity.

22. The suture of claim 21, wherein the porous interior component comprises a braided structure of multiple filaments, at least some of the filaments being hydrophilic and at least some of the filaments being hydrophobic.

23. The suture of claim 21, wherein the porous interior component comprises:
a porous interior core; and
an exterior component relative to the interior porous core,
wherein at least a portion of the interior porous core is hydrophilic and at least a portion of the exterior component is hydrophobic.

24. The suture of claim 21, wherein the plurality of the portions are along a length of the suture.

25. The suture of claim 21, wherein the suture comprises a hydrophilic portion sandwiched between at least two hydrophobic sections along a length of the suture.

26. The suture of claim 21, wherein the suture comprises a hydrophilic portion sandwiched between two hydrophobic section along a length of the suture, and wherein the first number of the biological cells are dispersed in the hydrophilic portion.
